(19) United States
(12) Patent Application Publication
(10) Pub. No.: US 2026/0204410 A1
(43) Pub. Date: Jul. 16, 2026

(54) Machine Learning Systems and Methods to Identify Locations of Potential Patients and/or Healthcare Providers

(71) Applicant: Genzyme Corporation, Cambridge, MA (US)

(72) Inventors: Sachin Mathur, Cambridge, MA (US); Youssef Meguebli, Cambridge, MA (US); Shannon Lee Smith, Cambridge, MA (US)

(21) Appl. No.: 19/128,502

(22) PCT Filed: Nov. 3, 2023
(86) PCT No.: PCT/IB2023/000775

§ 371 Date: May 8, 2025

Related U.S. Application Data
(60) Provisional application No. 63/424,250, filed on Nov. 10, 2022.

(30) Foreign Application Priority Data
Nov. 18, 2022 (EP) 22208436.0

(51) Int. Cl. G16H 50/20

(52) U.S. Cl. CPC G16H 50/20

(57) ABSTRACT

This specification relates to systems and methods for identifying locations of potential patients and/or healthcare providers from geofencing data using machine-learned models. According to a first aspect of this specification, there is described a computer implemented method of identifying potential disease diagnoses and/or healthcare providers. The method comprises: receiving geofencing data from one or more healthcare locations, the geofencing data relating to the disease comprising one or more impressions made at the healthcare location and/or one or more clicks made at the healthcare location; extracting, for each of a plurality of entities, a plurality of features from the geofencing data, wherein each entity is associated with a respective healthcare location in the one or more healthcare locations.

### CROSS REFERENCE TO RELATED APPLICATIONS

The present application is the national stage entry of International Patent Application No. PCT/IB2023/000775, filed on Nov. 3, 2023, and claims priority to Application No. EP 22208436.0, filed on Nov. 18, 2022, and U.S. Provisional Application No. 63/424,250, filed on Nov. 10, 2022 the disclosures of which are incorporated herein by reference.

### TECHNICAL FIELD

This specification relates to systems and methods for identifying locations of potential patients and/or healthcare providers from geofencing data using machine-learned models.

### BACKGROUND

Many diseases require timely intervention for better patient outcomes. Delays in treatment and diversity of symptoms can mask a diagnosis or lead to poor outcomes such as comorbidities. By identifying patient diagnoses early, patients may see an increase in health outcomes and disease management. Through timely, educational efforts, patients can potentially see a reduction in signs and symptoms and reduce other health risks.

Examples of such diseases may include some rare diseases. It is estimated that around 300 million people worldwide suffer from a rare disease. Rare diseases typically take longer to diagnose than more common diseases, with the average diagnosis time for a rare disease being over four years. This can lead to significant delays in treating the rare disease and a decrease in the likelihood of successfully treating the rare disease. Many factors contribute to this delay, including unfamiliarity with the rare disease by medical practitioners, a diversity of symptoms for a given rare disease and masking of the disease by symptoms of more common diseases. While traditional diagnostic algorithms can sometimes be effective, they rely on Health Care Providers (HCP) awareness, and require verifying numerous clinical characteristics, including differential diagnoses. Such conditions are rarely met in the real world.

### SUMMARY

A machine-learned computer implemented method of identifying potential disease diagnoses and/or healthcare institutions seeking education on specific disease diagnoses through geofencing data from one or more healthcare locations relating to the disease comprising one or more impressions and/or one or more clicks made at the healthcare location in combination with historical patterns from claims or laboratory findings and historical sales/prescription sales order data.

According to a first aspect of this specification, there is described a computer implemented method of identifying potential disease diagnoses and/or healthcare providers. The method comprises: receiving geofencing data from one or more healthcare locations, the geofencing data relating to the disease comprising one or more impressions made at the healthcare location and/or one or more clicks made at the healthcare location; extracting, for each of a plurality of entities, a plurality of features from the geofencing data, wherein each entity is associated with a respective healthcare location in the one or more healthcare locations; processing, using a machine-learned model, the extracted features for each entity to determine an indication of whether a potential disease patient is present at the respective healthcare location associated with that entity and/or if a healthcare provider at the respective healthcare location associated with that entity is potentially seeking information on the disease; and in response to determining an indication that a potential disease patient is present at a healthcare location in the one or more healthcare locations and/or that a healthcare provider at the respective healthcare location associated with that entity is potentially seeking information on the disease, triggering an alert, the alert comprising the identity of said health care location and/or the entity associated with said healthcare location.

This, and other aspects, may further include one or more of the following features, either alone or in combination.

The plurality of features may comprise: a number of impressions in a first time period; a number of impressions in a second time period; a number of clicks in the first time period; and a number of clicks in the second time period.

The method may further comprise receiving one or more sets of lab test data relating to the disease from one or more of the healthcare locations, each set of lab test data indicative of which lab tests have been performed by an entity at the healthcare location. The plurality of features may further be extracted from the one or more sets of lab test data. The plurality of features may comprise: a number of lab orders and/or lab tests in a first time period; and a number of lab orders and/or lab tests in a second time period. The first time period may be between 3 and 5 days, and the second time period may be between 5 and 14 days, e.g. 10 days.

The method may be repeated periodically, wherein, at each repetition, extracting the plurality of features comprises extracting a plurality of features from geofencing data captured within a predefined period of time prior to the time of the repetition.

The disease may be a rare disease or ultra-rare disease.

Processing the extracted features for each entity to determine the indication of whether a potential individual with a disease is present at the respective healthcare location associated with that entity may comprise, for each entity: determining a probability that a disease patient is present at the respective healthcare location associated with the entity using the machine-learned model and/or that a healthcare provider at the respective healthcare location associated with that entity is potentially seeking information on the disease; comparing the determined probability to a threshold probability level; and in response to determining that the determined probability is greater than the threshold probability level, indicating that a potential disease patient is present at the respective healthcare location associated with the entity and/or that a healthcare provider at the respective healthcare location associated with that entity is potentially seeking information on the disease.

Triggering the alert may comprise transmitting, to a healthcare professional at the identified healthcare location, educational material relating to the disease. Triggering the alert may comprise transmitting, to a sales representative, an indication that a potential disease diagnoses or information about the disease is associated with a health care facility/entity.

The method may further comprise: receiving search data relating to one or more search terms related to the disease; and extracting, for each of the plurality of entities, one or more features from the search data.

The machine-learned model may comprise: a logistic regression model; a random forest model; a neural network; a Generalised Additive Model; and/or a XGBoost model.

The plurality of entities may comprise one or more of: one or more customer accounts associated with a respective healthcare location; one or more departments and/or sub-departments associated with a respective healthcare location; one or more healthcare professionals associated with a respective healthcare location; and/or one or more healthcare providers associated with a respective healthcare location.

According to a further aspect of this specification, there is described a computer program product comprising computer readable instructions that, when executed by a computer, causes the computer to perform any one or more of the methods disclosed herein.

According to a further aspect of this specification, there is described a system comprising one or more processors and a memory, the memory storing computer readable instructions that, when executed by the one or more processors, causes the computer to perform any one or more of the methods disclosed herein.

As used herein, the term “rare disease” is preferably used to connote a disease that affects fewer than 1 in 2000 people within the general population. Currently, there are over 6,000 known rare diseases, and new rare diseases are being discovered all the time. The term “rare disease” may also encompass ultra-rare diseases. The term “ultra-rare disease” is preferably used to connote a disease that affects fewer than 1 in 50,000 people within the general population. However, the methods, systems and apparatus described herein are not limited to use with rare and ultra-rare diseases, and may be applied to disease of any type/rarity.

### BRIEF DESCRIPTION OF THE FIGURES

Example implementations will be described by way of reference to the accompanying drawings, in which:

FIG. 1 shows a schematic overview of a method for identifying locations where a potential disease patient may be present;

FIG. 2 shows a schematic overview of a method 200 for training a machine-learning model to identify locations where a potential disease patient may be present;

FIG. 3 shows a flow diagram of an example method of identifying potential disease diagnoses; and

FIG. 4 shows a schematic example of a system/apparatus 400 for performing any of the methods described herein.

### DETAILED DESCRIPTION

The methods and systems described herein utilize machine-learning to identify healthcare locations at which individuals with a disease (also referred to herein as disease patients) may be present from geofencing data associated with the healthcare locations. This allows alerts to be issued indicating the potential presence of disease patient, and additional actions to be taken accordingly, such as the provision of information about the disease. This can result in the diagnosis and treatment of a disease patient occurring more quickly, increasing the likelihood of a successful management of the disease. In some embodiments, the disease may be a rare disease or ultra-rare disease, though the methods and systems described herein may equally be applied to non-rare diseases.

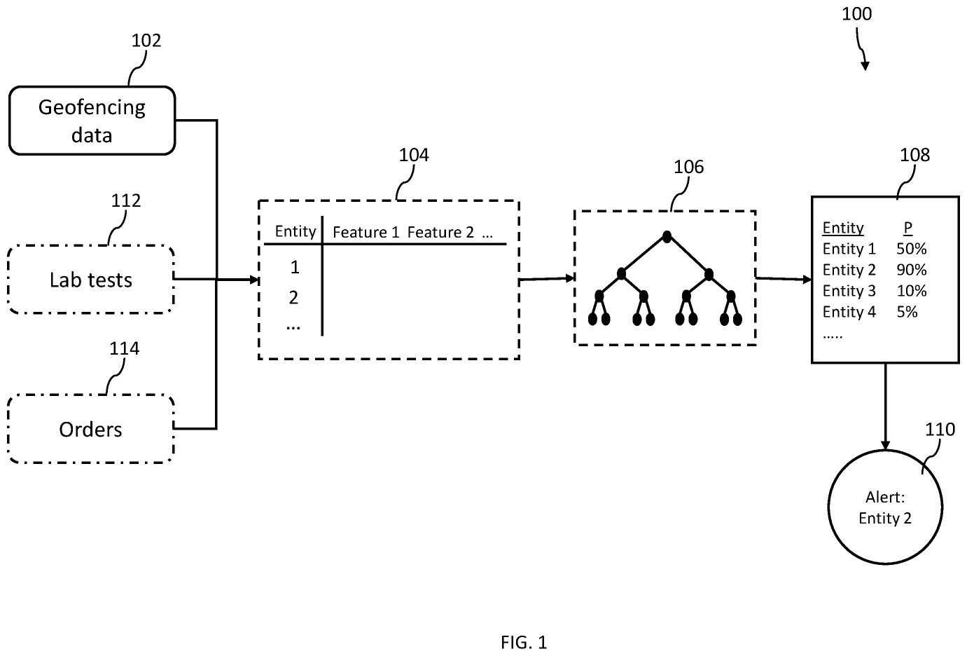

FIG. 1 shows a schematic overview of a method 100 for identifying locations where a potential disease patient may be present, and/or locations at which a healthcare provider/professional may be seeking information about a disease.

The method comprises receiving geofencing data 102 relating to a plurality of healthcare locations, and extracting features 104 from the geofencing data for each of a plurality of entities associated with the healthcare locations/sub-location. The extracted features are input into one or more machine-learned models 106, which process them to generate a prediction 108 relating to whether an individual having a disease, for instance a rare disease, is present at the location/sub-location associated with each entity. If such a patient (or a plurality of such patients) is present at one or more locations/sub-locations, then an alert 110 is issued identifying the one or more entities associated with the location. The method 100 may be performed/iterated periodically, e.g. repeated every day.

A geofence comprises a virtual boundary defining a real-world area. It may be based on defined boundaries/a footprint of a real-work location (e.g. a hospital), a based on a distance from a predetermined location (e.g. within 10 m of a diagnostic department in a hospital), or based on a predetermined travel time from a predetermined location (e.g. within 10 minutes travel time of hospital). In the methods described herein, geofences are constructed around healthcare locations/sub-locations Examples of such locations/sub-locations include: hospitals; hospital departments; pharmacies; family doctor locations or the like.

When a user of a mobile computing device crosses into a geofence area, the presence of the user in the geofence area is detected. For example, the GPS location of the user may be detected to be within the geofenced area and/or the location of a wireless access point used by the user may be within the geofenced area. Based on a user being within a geofenced area, advertisements and/or information relating to a given disease may be served to the user.

Geofencing data 102 comprises information relating to the user interaction with the advertisements and/or information served in a search engine to the user in a geofenced location.

The geofencing data may comprise “impression” data for each geofenced location. Each instance of serving an advert/set of information may be referred to as an “impression”. The geofencing data may comprise a number of times a set of adverts/information was served to users in a geofenced location over one or more time periods, i.e. the number of impressions. For example, the geofencing data 102 may comprise a number of impressions relating to a particular disease on each day for a predefined number of preceding days.

The geofencing data 102 may comprise “click” data for each geofenced location. Each time a user interacts with a served advert/set of information generates a click. For example, each time a user clicks on/selects/interacts with a served advert or set of information, a “click” is generated. The geofencing data may comprise a number of clicks related to a set of adverts/information served to users in a geofenced location over one or more time periods. For example, the geofencing data 102 may comprise a number of clicks relating to a particular disease on each day for a predefined number of preceding days.

The geofencing data 102 may further comprise search term data for each geofenced location. The search term data may comprise a count of the number of times one or more terms relating to the disease have been searched within a geofenced location.

The search terms may, for example, comprise the name (or names) of the disease, symptoms of the disease, treatments for the disease or the like. Search terms may be grouped into one or more collections of similar and/or closely related terms, for example synonyms.

The method 100 may use geofencing data 102 collected from a predefined rolling time period prior to the method 100 being performed. For example, the geofencing data 102 may be geofencing data 102 collected during the preceding 14 days.

For each of a plurality of entities, each corresponding to a geofenced location, a plurality of features 104 are extracted from the geofencing data 102. The plurality of features 104 may comprise, for example, a number of impressions associated with each entity in each of one or more time periods, e.g. the previous 3, 5, 7, 10 and/or 14 days. The plurality of features 104 may comprise, for example, a number of clicks associated with each entity in each of one or more time periods, e.g. the previous 3, 5, 7, 10 and/or 14 days. The plurality of features 104 may comprise, for example, a number of times each search term (or each collection of similar search terms) was input at the geofenced location associated with each entity in each of one or more time periods, e.g. the previous 3, 5, 7, 10 and/or 14 days. Additional features may be extracted from further input data, as described below.

Each geofenced healthcare location may have one or more entities associated with it. The entity may, for example, be the healthcare location itself, e.g. the identity of the hospital associated with the geofenced location, or a department/sub-department within a hospital. Alternatively or additionally, the one or more entities associated with a location may comprise one or more accounts associated with the healthcare location, e.g. commercial accounts. The one or more entities associated with a healthcare location may alternatively or additionally comprise the identity of a healthcare professional or provider associated with the location, such as the name of a doctor and/or the healthcare provider's National Provider Identifier (NPI) working at that location if they are opted in to receive advertisements.

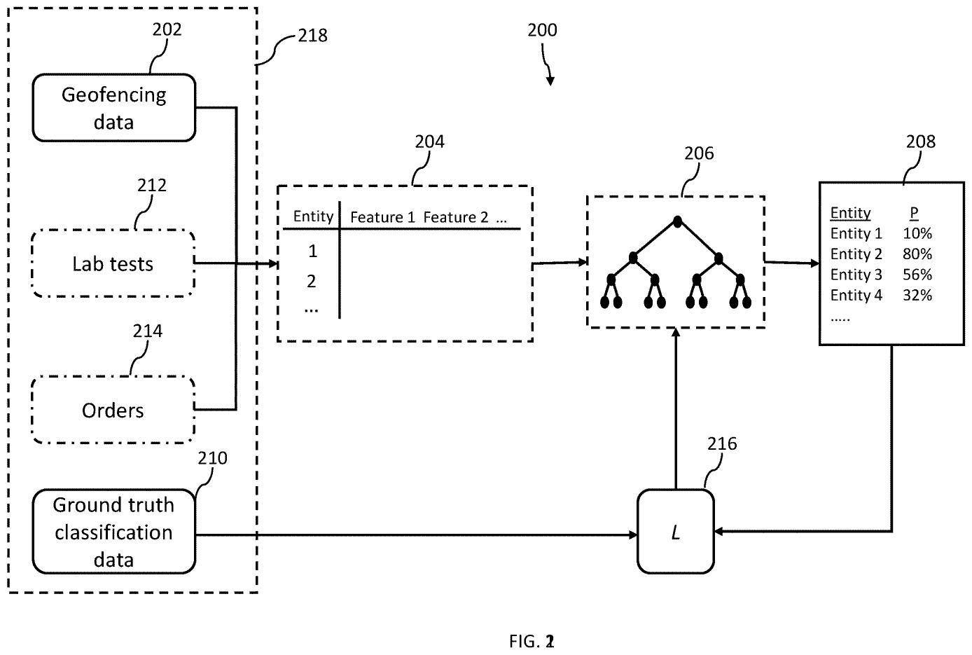

The one or more machine-learned models 106 comprise one or more parametrized models that have been trained to predict data indicative of the presence of a potential disease patient 108 at a healthcare location in the time period from which the geofencing data 102 was collected based on the extracted features 104. Examples of training processes are described below in relation to FIG. 2. The one or more machine learning models 106 take as input the extracted features 104 and process it based on the learned parameters of the models to generate an output indicative of the presence of a potential disease patient 108. The output 108 may, for example, comprise a probability of a disease patient being present at each healthcare location/entity, and/or a probability of a healthcare provider desiring information about a disease (e.g. symptoms, causes, treatments etc.). The probability may be a score between zero and one, or a percentage score. Alternatively, the output may be a binary indication of the presence of a potential disease patient or healthcare provider seeking information, e.g. a one indicating the presence of a potential disease patient and a zero indicating the absence of any potential disease patient.

Processing the extracted data 104 using the one or more machine-learned models 106 may comprise individually inputting extracted features for each entity into a machine-learned model 106 in sequence. The machine-learned model 106 processes the input data for each entity individually to produce the prediction for that entity, then receives input data for the next entity.

Alternatively, for N entities, N copies of a machine-learned model 106 may be used to process the extracted features 104 for the entities in parallel. Each copy of the machine-leaned model 106 processes the extracted features 104 from a respective entity, and outputs the respective prediction 108 for that entity.

The one or more machine-learned models 106 may, for example, comprise: a random forest model; a logistic regression model; an XGBoost model; a neural network, such as a fully connected neural network or the like; and/or a generalized additive model (GAM).

If the output 108 of the one or more machine-learned models 106 indicates the presence of a potential disease patient at a location associated with an entity and/or a healthcare provider at the respective healthcare location associated with that entity is potentially seeking information on the disease, then an alert 110 is triggered. The output 108 of the one or more machine-learned models 106 may be compared to a threshold condition to determine whether to trigger an alert 110. For example, the probability of a disease patient being present at a location may be compared to a threshold probability. If the threshold probability is exceeded, then the alarm is triggered for that location or the one or more entities associated with that location. The threshold

probability may, for example, be equivalent to a 70%, 80% or 90% probability.

The alerts 110 comprise an identifier of the entities and/or locations at which a potential disease patient has been detected and/or where a healthcare provider at the respective healthcare location associated with that entity is potentially seeking information on the disease. The alerts 110 may further comprise additional data associated with the detection, such as a time/date of the detections and/or the contact details of the entities where a potential disease patient has been detected. Alerts 110 may be transmitted to the entities associated with the locations where potential disease patients/interested healthcare providers are detected, and/or to one or more third parties, such as a research institute studying the disease and/or a manufacturer/distributor of treatments for the disease. In some implementations, the alerts may be provided to a sales or marketing representative of a pharmaceutical company, and act as a form of lead generation.

The alerts 110 may trigger one or more further actions. For example, in some implementations, an alert 110 may trigger the transmission of information relating to the disease to be sent to the identified entities and/or healthcare professionals associated with said entities. In this way, healthcare professionals at locations where a potential disease patient is present are educated and/or reminded about properties of the disease, thereby increasing the likelihood of a correct diagnosis.

In some implementations, when an alert 110 is triggered for an entity, the method 100 may comprise determining whether a previous alert for the entity has been triggered in a predetermined time period prior to the present alert. If an alert has been triggered within that time period, then the current alert 110 for that entity may be discarded and/or ignored. The predetermined time period may, for example be seven days.

In some implementations, alerts may be stored and analyzed to develop insights and identify trends in disease-related information. For example, clusters of potential patients with a given disease may be identified. Information and/or marketing activities relating to that disease may then be targeted at locations associated with the clusters. The insights/trends may trigger further alerts/actions. For example, where a disease has an underlying environmental cause, clusters of potential disease patients may trigger an alert that said environmental condition may be present in a geographic area.

The extracted features 104 may additionally be extracted from one or more further sets of input data associated with each geofenced location. An example of such data may be the number and type of lab tests 112 performed by entities associated with each geofenced location. Such data may be anonymized, and comprise a number of times each of a plurality of different lab tests associated with the disease have been performed that originated from entities associated with the geofenced location, e.g. ordered by those entities. Another example, the one or more further sets of input data associated with each geofenced location may alternatively or additionally comprise lab orders 114 made by the entities associated with the geofenced locations.

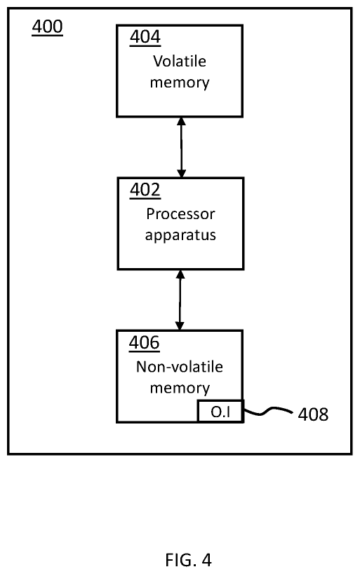

FIG. 2 shows a schematic overview of a method 200 for training a machine-learning model to identify locations where a potential disease patient may be present and/or locations where a healthcare provider is potentially interested in information about a given disease. The method may be performed by one or more computer systems such as the system described below in relation to FIG. 4.

The training method 200 uses training examples 218 taken from a set of training data. Each example comprises a set of input data, comprising historic geofencing data 202, and a corresponding ground truth classification 210 for the input data. The geofencing data may the geofencing data described above in relation to FIG. 1. The input data may further comprise additional data types, such as the number and type of lab tests 112 performed by entities associated with each geofenced location and/or lab orders 114 made by the entities associated with the geofenced locations.

The ground truth classification 210 comprises data indicative of whether a disease patient was present at a healthcare location associated with the geofencing data 202 during the period covered by the geofencing data 202. Such data may comprise an indication of a positive diagnoses of the disease at the healthcare location within a predetermined time period of the period covered by the geofencing data, e.g. within seven days.

Alternatively or additionally, the ground truth classification 210 may comprise an indirect indication of a disease patient being present, such an order from/sale to an entity associated with the healthcare location for a drug/equipment for treating the disease within a predetermined time period of the period covered by the geofencing data, e.g. within seven days. Treatment sales/orders within a predetermined geodesic distance (e.g. 1 km) of a geofenced location may be counted as a originating from the healthcare location associated with that geofenced location. This can increase the amount of training data available, resulting an increased performance of the machine-learning models 206 once trained.

During training, a plurality of features 204 for each entity of a plurality of entities is extracted from the input data. The extracted features 204 for each entity are processed by one or more machine-learning models 206 to generate a candidate prediction 208 for the presence of a disease patient at the healthcare location associated with each entity. The candidate predictions 208 are compared to corresponding ground truth classifications 210 using a loss/objective function 216. The loss function 216 may comprise an area under curve metric. Alternatively, a classification loss, such as binary cross entropy, may be used. The value of the loss/objective function 216 is used to generate updates to the machine-leaning model 206 using an optimization routine, e.g. stochastic gradient descent.

The training may be iterated over the training data until a threshold condition is satisfied. The threshold condition may be a threshold number of iterations/epochs of training, and/or a threshold performance being reached on a test dataset. The test dataset may have the same structure as the training dataset, but with different examples included.

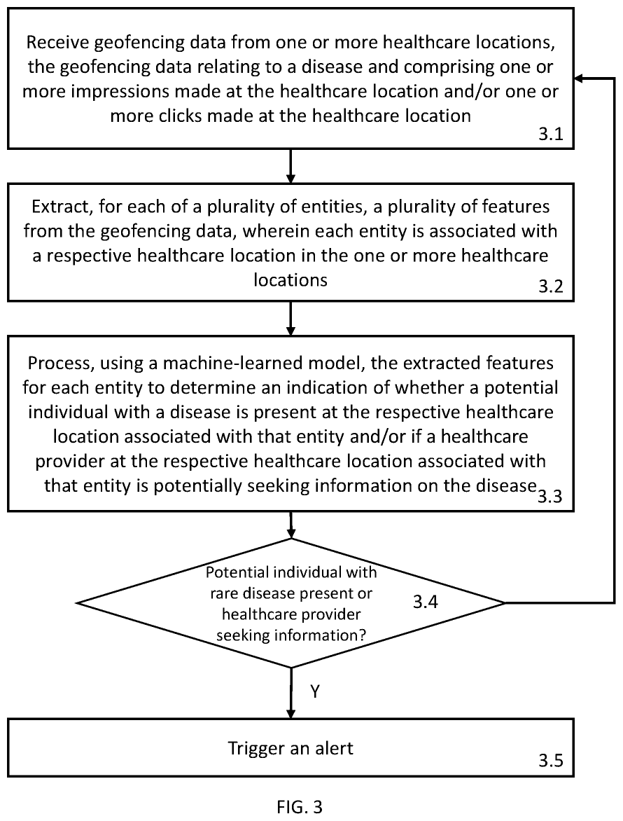

FIG. 3 shows a flow diagram of an example method of identifying potential disease diagnoses and/or identifying healthcare providers who may have interest in a given disease. The method may be performed by one or more computing systems/apparatus, such as the systems/apparatus described in relation to FIG. 4.

At operation 3.1, geofencing data is received from one or more healthcare locations. The geofencing data comprises one or more impressions relating to the disease made at the healthcare location and/or one or more clicks relating to the disease made at the healthcare location.

In some embodiments, one or more further inputs are also received. The one or more further inputs may comprise search data relating to one or more search terms related to the disease, e.g. a number of times that each of one or more search terms has been searched at each healthcare location. The search terms may, for example, comprise symptoms of the disease, treatments of the disease and/or names/types of the disease. The one or more further inputs may alternatively or additionally comprise one or more sets of lab test data relating to the disease from one or more of the healthcare locations. Each set of lab test data may be indicative of which of a plurality of lab tests have been performed by an entity at the healthcare location, e.g. how many blood tests of a particular type have been performed at a healthcare location and/or by an entity.

At operation 3.2, a plurality of features are extracted from the geofencing data for each of a plurality of entities. Each entity is associated with a respective healthcare location in the one or more healthcare locations. Features may also be extracted from any additional inputs to the method.

The plurality of features comprises: a number of impressions in a first time period; a number of impressions in a second time period; a number of clicks in the first time period; and a number of clicks in the second time period. The first time period may be between 3 and 5 days, e.g. 3 days, and the second time period may be between 5 and 14 days, e.g. 5 days.

The plurality of features may further comprise a number of impressions in a third time period and/or a number of clicks in a third time period. The third time period may be between 7 and 14 days, e.g. 10 days. The features may further comprise a number of impressions in a fourth time period and/or a number of clicks in a fourth time period. The fourth time period may be between 12 and 21 days, e.g. 14 days.

In implementations where lab order data and/or lab tests are also received, the extracted features may further comprise a number of lab orders and/or lab tests in the first time period, second time period, third time period and/or fourth time period.

The plurality of entities comprises one or more of: one or more customer accounts associated with a respective healthcare location; one or more departments and/or sub-departments associated with a respective healthcare location; one or more healthcare professionals associated with a respective healthcare location; and/or one or more healthcare providers associated with a respective healthcare location.

At operation 3.3, the extracted features for each entity are processed using one or more machine-learned models, which outputs data indicative of whether a potential individual with a disease is present at the respective healthcare location associated with that entity and/or if a healthcare provider at the respective healthcare location associated with that entity is potentially seeking information relating to the disease.

The output of the machine learning model may be a probability for each entity/location that an individual with the disease is present.

The machine-learned model may comprise one or more of: a logistic regression model; a random forest model; a GAM; a neural network (e.g. a fully connected neural network); and/or an XGBoost model. The machine-learned model may have been trained on historic geofencing data, as described above in relation to FIG. 2.

At operation 3.4, the potential presence of a disease patient and/or a healthcare provider at the respective healthcare location associated with that entity is potentially seeking information on the disease (e.g. symptoms, diagnostic information/tests and/or treatments) is assessed based on the data output by the machine-learned model. If the data indicate that an individual with a disease is potentially present and/or a healthcare provider at the respective healthcare location associated with that entity is potentially seeking information relating to the disease, then the method proceeds to operation 3.5. Otherwise, the method returns to operation 3.1 and awaits the next batch of geofencing data.

The presence or absence of a potential disease patient and/or healthcare provider at the respective healthcare location associated with that entity is potentially seeking information relating to the disease may be determined by comparing the data output by the machine-learned model to one or more thresholds. For example, each entities probability of having an individual with the disease may be compared to a threshold probability (e.g. 70%, 80% or 90%, among others). If the probability of having an individual with the disease present for an entity exceeds the threshold probability, then it is determined that a potential disease patient may be present at the location associated with that entity.

At operation 3.5, an alert is triggered. The alert comprises the identity of said health care location and/or the entity associated with said healthcare location. The method then returns to operation 3.1 and awaits the next batch of geofencing data.

The alert may be sent to a healthcare professional at the identified healthcare location (i.e. the location associated with the identified entity). The alert may comprise educational material relating to the disease, such as symptoms of the disease and/or treatments/therapies for the disease. In some implementations, the alert may comprise a link to a site where a medicament for the disease may be ordered.

Alternatively or additionally, the alert may be sent to a sales or marketing representative at a pharmaceutical company that produces treatments for the disease.

Batches of geofencing data (and, in some implementations, other input data) may be received periodically, for example once each day. The method may be repeated each time a new batch is received. At each repetition, extracting the plurality of features may comprise extracting a plurality of features from geofencing data captured within a predefined period of time prior to the time the new data is received. This predefined period may be greater than 10 days, e.g. 14 days.

FIG. 4 shows a schematic example of a system/apparatus 400 for performing any of the methods described herein. The system/apparatus shown is an example of a computing device. The system/apparatus 400 may form at least a part of a concrete mixer, e.g. part of an ECU of a concrete mixer.

The apparatus (or system) 400 comprises one or more processors 402. The one or more processors control operation of other components of the system/apparatus 400. The one or more processors 402 may, for example, comprise a general-purpose processor. The one or more processors 402 may be a single core device or a multiple core device. The one or more processors 402 may comprise a Central Processing Unit (CPU) or a graphical processing unit (GPU). Alternatively, the one or more processors 402 may comprise specialized processing hardware, for instance a RISC processor or programmable hardware with embedded firmware. Multiple processors may be included.

The system/apparatus comprises a working or volatile memory 404. The one or more processors may access the volatile memory 404 in order to process data and may control the storage of data in memory. The volatile memory 404 may comprise RAM of any type, for example, Static RAM (SRAM) or Dynamic RAM (DRAM), or it may comprise Flash memory, such as an SD-Card.

The system/apparatus comprises a non-volatile memory 406. The non-volatile memory 406 stores a set of operation instructions 408 for controlling the operation of the processors 402 in the form of computer readable instructions. The non-volatile memory 406 may be a memory of any kind such as a Read Only Memory (ROM), a Flash memory or a magnetic drive memory.

The one or more processors 402 are configured to execute operating instructions 408 to cause the system/apparatus to perform any of the methods described herein. The operating instructions 408 may comprise code (i.e. drivers) relating to the hardware components of the system/apparatus 400, as well as code relating to the basic operation of the system/apparatus 400. Generally speaking, the one or more processors 402 execute one or more instructions of the operating instructions 408, which are stored permanently or semi-permanently in the non-volatile memory 406, using the volatile memory 404 to store temporarily data generated during execution of said operating instructions 408.

Any mentioned apparatus and/or other features of particular mentioned apparatus may be provided by apparatus arranged such that they become configured to carry out the desired operations only when enabled, e.g. switched on, or the like. In such cases, they may not necessarily have the appropriate software loaded into the active memory in the non-enabled (e.g. switched off state) and only load the appropriate software in the enabled (e.g. on state). The apparatus may comprise hardware circuitry and/or firmware. The apparatus may comprise software loaded onto memory. Such software/computer programs may be recorded on the same memory/processor/functional units and/or on one or more memories/processors/functional units.

Any mentioned apparatus/circuitry/elements/processor may have other functions in addition to the mentioned functions, and that these functions may be performed by the same apparatus/circuitry/elements/processor. One or more disclosed aspects may encompass the electronic distribution of associated computer programs and computer programs (which may be source/transport encoded) recorded on an appropriate carrier (e.g. memory, signal).

Any “computer” described herein can comprise a collection of one or more individual processors/processing elements that may or may not be located on the same circuit board, or the same region/position of a circuit board or even the same device. In some examples one or more of any mentioned processors may be distributed over a plurality of devices. The same or different processor/processing elements may perform one or more functions described herein.

The term “signaling” may refer to one or more signals transmitted as a series of transmitted and/or received electrical/optical signals. The series of signals may comprise one, two, three, four or even more individual signal components or distinct signals to make up said signaling. Some or all of these individual signals may be transmitted/received by wireless or wired communication simultaneously, in sequence, and/or such that they temporally overlap one another.

With reference to any discussion of any mentioned computer and/or processor and memory (e.g. including ROM, CD-ROM etc.), these may comprise a computer processor, Application Specific Integrated Circuit (ASIC), field-programmable gate array (FPGA), and/or other hardware components that have been programmed in such a way to carry out the inventive function.

The applicant hereby discloses in isolation each individual feature described herein and any combination of two or more such features, to the extent that such features or combinations are capable of being carried out based on the present specification as a whole, in the light of the common general knowledge of a person skilled in the art, irrespective of whether such features or combinations of features solve any problems disclosed herein, and without limitation to the scope of the claims. The applicant indicates that the disclosed aspects/examples may consist of any such individual feature or combination of features. In view of the foregoing description, it will be evident to a person skilled in the art that various modifications may be made within the scope of the disclosure.

While there have been shown and described and pointed out fundamental novel features as applied to examples thereof, it will be understood that various omissions and substitutions and changes in the form and details of the devices and methods described may be made by those skilled in the art without departing from the scope of the disclosure. For example, it is expressly intended that all combinations of those elements and/or method steps which perform substantially the same function in substantially the same way to achieve the same results are within the scope of the disclosure. Moreover, it should be recognized that structures and/or elements and/or method steps shown and/or described in connection with any disclosed form or examples may be incorporated in any other disclosed or described or suggested form or example as a general matter of design choice. Furthermore, in the claims means-plus-function clauses are intended to cover the structures described herein as performing the recited function and not only structural equivalents, but also equivalent structures.

Implementations of the methods described herein may be realized as in digital electronic circuitry, integrated circuitry, specially designed ASICs (application specific integrated circuits), computer hardware, firmware, software, and/or combinations thereof. These may include computer program products (such as software stored on e.g. magnetic discs, optical disks, memory, Programmable Logic Devices) comprising computer readable instructions that, when executed by a computer, such as that described in relation to FIG. 7, cause the computer to perform one or more of the methods described herein.

Any system feature as described herein may also be provided as a method feature, and vice versa. As used herein, means plus function features may be expressed alternatively in terms of their corresponding structure. In particular, method aspects may be applied to system aspects, and vice versa.

Furthermore, any, some and/or all features in one aspect can be applied to any, some and/or all features in any other aspect, in any appropriate combination. It should also be appreciated that particular combinations of the various features described and defined in any aspects of the specification can be implemented and/or supplied and/or used independently.

Although several embodiments have been shown and described, it would be appreciated by those skilled in the art that changes may be made in these embodiments without departing from the principles of this disclosure, the scope of which is defined in the claims and their equivalents.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about-4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codeable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin. Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091 March-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrome. Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present specification include, for example, Fab fragments,

F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen. Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts. Those of skill in the art will understand that modifications (additions and/or removals) of various components of the APIs, formulations, apparatuses, methods, systems and embodiments described herein may be made without departing from the full scope and spirit of the present specification, which encompass such modifications and any and all equivalents thereof.

An example drug delivery device may involve a needle-based injection system as described in Table 1 of section 5.2 of ISO 11608-1:2014 (E). As described in ISO 11608-1:2014 (E), needle-based injection systems may be broadly distinguished into multi-dose container systems and single-dose (with partial or full evacuation) container systems. The container may be a replaceable container or an integrated non-replaceable container.

As further described in ISO 11608-1:2014 (E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014 (E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014 (E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

1-15. (canceled)

16. A computer implemented method of identifying potential disease diagnoses and/or healthcare providers, the method comprising:
receiving geofencing data from one or more healthcare locations, the geofencing data relating to the disease comprising one or more impressions made at the healthcare location and/or one or more clicks made at the healthcare location;
extracting, for each of a plurality of entities, a plurality of features from the geofencing data, wherein each entity is associated with a respective healthcare location in the one or more healthcare locations;
processing, using a machine-learned model, the extracted features for each entity to determine an indication of whether a potential disease patient is present at the respective healthcare location associated with that entity and/or if a healthcare provider at the respective healthcare location associated with that entity is potentially seeking information relating to the disease; and
in response to determining an indication that a potential disease patient is present at a healthcare location in the one or more healthcare locations and/or that a healthcare provider at the respective healthcare location associated with that entity is potentially seeking information relating to the disease, triggering an alert, the alert comprising the identity of said health care location and/or the entity associated with said healthcare location.

17. The method of claim 16, wherein the plurality of features comprises: a number of impressions in a first time period; a number of impressions in a second time period; a number of clicks in the first time period; and a number of clicks in the second time period.

18. The method of claim 16, further comprising:
receiving one or more sets of lab test data relating to the disease from one or more of the healthcare locations, each set of lab test data indicative of which lab tests have been performed by an entity at the healthcare location;
wherein the plurality of features is further extracted from the one or more sets of lab test data.

19. The method of claim 18, wherein the plurality of features comprises: a number of lab orders and/or lab tests in a first time period; and a number of lab orders and/or lab tests in a second time period.

20. The method of claim 17, wherein the first time period is between 3 and 5 days and the second time period is between 5 and 14 days.

21. The method claim 16, wherein the method is repeated periodically, wherein, at each repetition, extracting the plurality of features comprises
extracting a plurality of features from geofencing data captured within a predefined period of time prior to the time of the repetition.

22. The method of claim 16, wherein the disease is a rare disease or ultra-rare disease.

23. The method of claim 16, wherein processing the extracted features for each entity to determine the indication of whether a potential individual with a disease is present at the respective healthcare location associated with that entity comprises, for each entity:
determining a probability that a disease patient is present at the respective healthcare location associated with the entity using the machine-learned model and/or that a healthcare provider at the respective healthcare location associated with that entity is potentially seeking information relating to the disease;
comparing the determined probability to a threshold probability level; and
in response to determining that the determined probability is greater than the threshold probability level, indicating that a potential disease patient is present at the respective healthcare location associated with the entity and/or that a healthcare provider at the respective healthcare location associated with that entity is potentially seeking information relating to the disease.

24. The method of claim 16, wherein triggering the alert comprises transmitting, to a healthcare professional at the identified healthcare location, educational material relating to the disease.

25. The method of claim 16, wherein triggering the alert comprises transmitting, to a sales representative, an indication that a potential disease diagnoses or information about the disease is associated with a healthcare facility entity.

26. The method of claim 16, further comprising:
receiving search data relating to one or more search terms related to the disease; and
extracting, for each of the plurality of entities, one or more features from the search data.

27. The method of claim 16, wherein the machine-learned model comprises: a logistic regression model; a random forest model; a neural network; a generalized additive model; and/or a XGBoost model.

28. The method of claim 16, wherein the plurality of entities comprises one or more of: one or more customer accounts associated with a respective healthcare location; one or more departments and/or sub-departments associated with a respective healthcare location; one or more healthcare professionals associated with a respective healthcare location; and/or one or more healthcare providers associated with a respective healthcare location.

29. One or more non-transitory computer storage media storing instructions that when executed by one or more computers cause the one or more computers to perform operations for identifying potential disease diagnoses and/or healthcare providers, the operations comprising:
receiving geofencing data from one or more healthcare locations, the geofencing data relating to the disease comprising one or more impressions made at the healthcare location and/or one or more clicks made at the healthcare location;
extracting, for each of a plurality of entities, a plurality of features from the geofencing data, wherein each entity is associated with a respective healthcare location in the one or more healthcare locations;
processing, using a machine-learned model, the extracted features for each entity to determine an indication of whether a potential disease patient is present at the respective healthcare location associated with that entity and/or if a healthcare provider at the respective healthcare location associated with that entity is potentially seeking information relating to the disease; and
in response to determining an indication that a potential disease patient is present at a healthcare location in the one or more healthcare locations and/or that a healthcare provider at the respective healthcare location associated with that entity is potentially seeking information relating to the disease, triggering an alert, the alert comprising the identity of said health care location and/or the entity associated with said healthcare location.

30. A system comprising:
one or more computers; and
one or more storage devices communicatively coupled to the one or more computers, wherein the one or more storage devices store instructions that, when executed by the one or more computers, cause the one or more computers to perform operations for identifying potential disease diagnoses and/or healthcare providers, the operations comprising:
receiving geofencing data from one or more healthcare locations, the geofencing data relating to the disease comprising one or more impressions made at the healthcare location and/or one or more clicks made at the healthcare location;
extracting, for each of a plurality of entities, a plurality of features from the geofencing data, wherein each entity is associated with a respective healthcare location in the one or more healthcare locations;
processing, using a machine-learned model, the extracted features for each entity to determine an indication of whether a potential disease patient is present at the respective healthcare location associated with that entity and/or if a healthcare provider at the respective healthcare location associated with that entity is potentially seeking information relating to the disease; and
in response to determining an indication that a potential disease patient is present at a healthcare location in the one or more healthcare locations and/or that a healthcare provider at the respective healthcare location associated with that entity is potentially seeking information relating to the disease, triggering an alert, the alert comprising the identity of said health care location and/or the entity associated with said healthcare location.

31. The system of claim 30, wherein the plurality of features comprises: a number of impressions in a first time period; a number of impressions in a second time period; a number of clicks in the first time period; and a number of clicks in the second time period.

32. The system of claim 30, wherein the operations further comprise:
receiving one or more sets of lab test data relating to the disease from one or more of the healthcare locations, each set of lab test data indicative of which lab tests have been performed by an entity at the healthcare location;
wherein the plurality of features is further extracted from the one or more sets of lab test data.

33. The system of claim 32, wherein the plurality of features comprises: a number of lab orders and/or lab tests in a first time period; and a number of lab orders and/or lab tests in a second time period.

34. The system of claim 31, wherein the first time period is between 3 and 5 days and the second time period is between 5 and 14 days.

35. The system claim 30, wherein the operations are repeated periodically, wherein, at each repetition, extracting the plurality of features comprises:
extracting a plurality of features from geofencing data captured within a predefined period of time prior to the time of the repetition.
